(19) United States
(12) Patent Application Publication
(10) Pub. No.: US 2025/0223568 A1
(43) Pub. Date: Jul. 10, 2025

(54) METHOD OF ORGANOID DERIVED PRODUCTION OF HUMAN EXTRACELLULAR MATRIX HYDROGELS

(71) Applicant: The Board of Trustees of The University of Alabama, Tuscaloosa, AL (US)

(72) Inventors: Alexandra DeVera Avera, Tuscaloosa, AL (US); Yonghyun Kim, Tuscaloosa, AL (US)

(21) Appl. No.: 19/011,037

(22) Filed: Jan. 6, 2025

Related U.S. Application Data
(60) Provisional application No. 63/618,035, filed on Jan. 5, 2024.

(51) Int. Cl. C12N 5/09; A61K 35/30

(52) U.S. Cl. CPC C12N 5/0693; A61K 35/30; C12N 2533/90; C12N 2537/10

(57) ABSTRACT

Disclosed herein is a method of extracting extracellular matrix (ECM) from organoids via decellularization and hydrogels formed from methods thereof. The hydrogels can be used in medical devices for regenerative medicine like tissue grafts, and research and development applications to enhance disease modeling utilizing 3D bioprinting or other hydrogel embedding processes. The hydrogels can also be used to enhance tissue culture flasks for cells by offering a softer environment than polystyrene for cell culture. The hydrogels can be used in drug and vaccine delivery as well.

### CROSS REFERENCE TO RELATED APPLICATIONS

This application claims the benefit of priority to U.S. Provisional Application No. 63/618,035, filed Jan. 5, 2024, which is incorporated by reference herein in its entirety.

### STATEMENT OF GOVERNMENT SUPPORT

This invention was made with government support under Grant No. 2000053 awarded by the National Science Foundation and Grant No. P200A210069 awarded by the Department of Education. The government has certain rights in the invention.

### BACKGROUND

Biologically derived extracellular matrix hydrogels are becoming increasingly popular due to its naturally occurring biochemical cues and complexity. However, the sources of biologically derived extracellular matrix (ECM) are limited, difficult to process, and have a major risk of endotoxin contamination causing immunogenic response in application. Decellularization of human tissues and human embryoid bodies derived from stem cells (SCs) to form hydrogels has been extensively studied and performed.

Glioblastoma (GBM) is a brain tumor derived from mutated astrocyte cells. Healthy brain tissue is ˜20%-(v/v) ECM to cells, while glioblastoma tissue is ˜48%-(v/v) ECM to cells. GBM ECM is rich in growth factors, biochemical cues for cellular attachment and migration, collagen IV, tenascin proteins, elastin proteins, and hyaluronic acid (HA). The ECM collagen IV, tenascins, elastins, and HAs play major roles in tissue regeneration and are difficult to harness from readily available tissues. Recently, GBO models were further improved by adopting Lancaster's cerebral organoid culture system (Klein et al., 2020; Lancaster et al., 2013; Ogawa et al., 2018). Furthermore, a GBO culture system preserving the parental tumor features using resected patient tissues for chimeric antigen receptor (CAR) T cell therapy was established within two months (Jacob et al., 2020). However, this method requires patient samples, is not scalable, and displays various subregional sectioned clone-derived GBOs. Thus, the infusion of CAR-T cells into GBOs expressing heterogeneous antigens needs to be further studied.

Thus, there is a need for a means of producing extracellular matrix at scale and in a shorter production time. These needs and others are at least partially satisfied by the present disclosure.

### SUMMARY

Disclosed herein is a method of extracting extracellular matrix (ECM) from organoids via decellularization and hydrogels formed thereof. The hydrogels can be used in medical devices for regenerative medicine like tissue grafts, and research and development applications to enhance disease modeling utilizing 3D bioprinting or other hydrogel embedding processes. The hydrogels can also be used to enhance tissue culture flasks for cells by offering a softer environment than polystyrene for cell culture. The hydrogels can be used in drug and vaccine delivery as well.

### BRIEF DESCRIPTION OF DRAWINGS

FIGS. 1A-1B depict a proposed organoid product (FIG. 1B) and a proposed method for making the organoid product (FIG. 1A).

FIG. 2 depicts immunofluorescence images of the proposed organoid product.

FIG. 3 depicts control organoids and decellularized organoids stained with hematoxylin (purple) and eosin (pink).

FIG. 4 depicts an organoid-derived extracellular matrix hydrogel.

FIGS. 5A-5C depict an example of ECM quantification and immunohistochemical visualization.

### DETAILED DESCRIPTION

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Accordingly, these terms are intended to not only cover the recited element(s) or step(s) but may also include other elements or steps not expressly recited. Furthermore, as used herein, the use of the terms “a”, “an”, and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. A range may be construed to include the start and the end of the range. For example, a range of 10% to 20% (i.e., range of 10%-20%) can includes 10% and also includes 20%, and includes percentages in between 10% and 20%, unless explicitly stated otherwise herein.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The term “culturing” refers to the in vitro propagation of cells or organisms on or in media of various kinds. It is understood that the descendants of a cell grown in culture may not be completely identical (i.e., morphologically, genetically, or phenotypically) to the parent cell. By “expanded” it is meant any proliferation or division of cells.

“Differentiation” describes the process whereby an unspecialized cell acquires the features of a specialized cell such as a heart, liver, or muscle cell. “Directed differentiation” refers to the manipulation of stem cell culture conditions to induce differentiation into a particular cell type. “Dedifferentiated” defines a cell that reverts to a less committed position within the lineage of a cell. As used herein, the term “differentiates” or “differentiated” defines a cell that takes on a more committed (“differentiated”) position within the lineage of a cell. As used herein, “a cell that differentiates into a mesodermal (or ectodermal or endodermal) lineage” defines a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal.

As used herein, “multipotent” cells are more differentiated than pluripotent cells but are not permanently committed to a specific cell type. Pluripotent cells therefore have a higher potency than multipotent cells.

As used herein, “pluripotent cells” means a population of cells capable of differentiating into all three germ layers and becoming any cell type in the body. Pluripotent cells express a variety of cell surface markers, have a cell morphology characteristic of undifferentiated cells and form teratomas when introduced into an immunocompromised animal, such as a SCID mouse. Teratomas typically contain cells or tissues characteristic of all three germ layers.

A “precursor” or “progenitor cell” intends to mean cells that have a capacity to differentiate into a specific type of cell. A progenitor cell may be a stem cell. A progenitor cell may also be more specific than a stem cell. A progenitor cell may be unipotent or multipotent. Compared to adult stem cells, a progenitor cell may be in a later stage of cell differentiation. An example of progenitor cell includes, without limitation, a progenitor nerve cell.

As used herein, “serum-free” means that neither the culture nor the culture medium contains serum or plasma, although purified or synthetic serum or plasma components (e.g., FGFs) can be provided in the culture in reproducible amounts as described below.

As used herein, “stem cell” defines an adult undifferentiated cell that can produce itself and a further differentiated progeny cell and under certain situations, give rise to specialized cells.

As used here, a “substantially pure population” means a population of derived mesenchymal cells that contains at least 99% mesenchymal cells. Cell purification can be accomplished by any means known to one of ordinary skill in the art. For example, a substantially pure population of cells can be achieved by growth of cells or by selection from a less pure population, as described herein.

As used herein, the term “organoid” refers to a three-dimensional cell cultures that incorporates some of the key features of the represented organ. Unless specified to the contrary, an organoid may be obtained from adult stem cell-containing tissue samples, single adult stem cells, or from the directed differentiation of pluripotent stem cells, multipotent stem cells, or totipotent stem cells.

To facilitate an understanding of the principles and features of various embodiments of the present invention, they are explained hereinafter with reference to their implementation in illustrative embodiments.

In one aspect, a hydrogel comprising an extracellular matrix (ECM) is described. The extracellular matrix is comprised of decellularized organoids, wherein the organoids are derived from human stem cells. In some aspects, the human stem cells are brain stem cells, in particular, glioblastoma stem cells. The hydrogel is formed by harvesting ECM from a sample of organoids and polymerizing the organoid-derived ECM.

The organoids are formed from human-derived stem cells, in particular glioblastoma stem cells, to produce highly heterogeneous ECM and hydrogels. Glioblastoma stem cells have a high proportion of ECM, making them an ideal source of organoid-derived ECM for the described hydrogel. In some aspects, the physical form of organoid-derived ECM can be additionally modified by crosslinking and sterilization to form a hydrogel.

In an exemplary aspect, a sample of organoid is decellularized following standard protocols (ADA-0-005-18-24,-79,-90-93, and ADA-0-006-10-11) including harvesting and preparing the organoid sample (e.g., centrifuging the sample, removing supernatants, resuspending in deionized water, and freezing the sample for storage and later use) and removing cellular components from the organoid sample (e.g., using a detergent solution), thereby isolating organoid-derived ECM. Additionally, the organoid sample is lyophilized before removing cellular components; alternatively, the organoid sample is not lyophilized. The method of decellularization can include adding Triton X-100 (TX100) decellularization solution to the sample. The TX100 sample may include TX100, ammonium hydroxide, and a protease inhibitor in deionized water. It should be noted that TX100, ammonium hydroxide, and a protease inhibitor are used here as examples and other reagents/detergents may be used to remove cellular components. After decellularizing, the sample is washed 2-3 times to remove cellular particulates from the remaining organoid-derived ECM. In some aspects, the washed organoid-derived ECM is lyophilized. The method to decellularize the organoid sample further includes providing an endonuclease solution (i.e. DNasel solution) and an endonuclease buffer solution including Tris-HCl, MgCl₂, and CaCl₂ in deionized water to the washed organoid-derived ECM, incubating at 37° C., centrifuging, and washing the sample 2-3 times. It should be understood that this aspect provides an exemplary method, and that alternatives, substitutions, or omissions of one or more steps are possible while still achieving a decellularized organoid sample.

In some aspects, the organoid-derived ECM is lyophilized. In some aspects, the lyophilized organoid-derived ECM is digested (e.g., by providing pepsin in an acidic environment) and, optionally, neutralized to pH 7.

In some aspects, a hydrogel is formed by polymerizing the organoid-derived ECM.

In some aspects, a hydrogel is formed by thermal polymerization of the organoid-derived ECM. Thermal polymerization may be suitable for forming hydrogels intended to be used for coating surfaces and/or quick encapsulation.

In some aspects, a hydrogel is formed by thermal, chemical, and/or light activated crosslinking. In some aspect, the hydrogel may be suitable for 3D printing applications.

It should be understood that alternative methods to the example aspects of forming hydrogels may be used to form a hydrogel for specific applications, needing specific properties. A hydrogel of the present disclosure is derived from decellularized organoids, specifically organoid-derived ECM.

It is contemplated that the hydrogels described herein may be used in a method of treatment for regenerating tissue (i.e. regenerative medicine). In some examples, the method may be for treatment of traumatic brain injury, spinal cord injury, diseases of the brain or spinal cord tissue, or may undergo additional post-decellularization to meet commercial needs for biopolymers for hard to regenerate tissues, such as tendons and nerves. The hydrogels can be used in medical devices for regenerative medicine such as tissue grafts, and research and development applications to enhance disease modeling utilizing 3D bioprinting or other hydrogel embedding processes. The hydrogels can also be used to enhance tissue culture flasks for cells by offering a softer environment than polystyrene for cell culture. The hydrogels can be used in drug and vaccine delivery as well.

In some aspects, glioblastoma stem cells are used to form glioblastoma organoids. Glioblastoma (GBM) is a brain tumor derived from mutated astrocyte cells. Healthy brain tissue is ˜20%-(v/v) ECM to cells, while glioblastoma tissue is ˜48%-(v/v) ECM to cells. GBM ECM is rich in growth factors, which are biochemical cues for cellular attachment and migration, collagen IV, tenascin proteins, elastin proteins, and hyaluronic acid (HA). The ECM collagen IV, tenascins, elastins, and HAs play major roles in tissue regeneration and are difficult to harness from state of the art tissues.

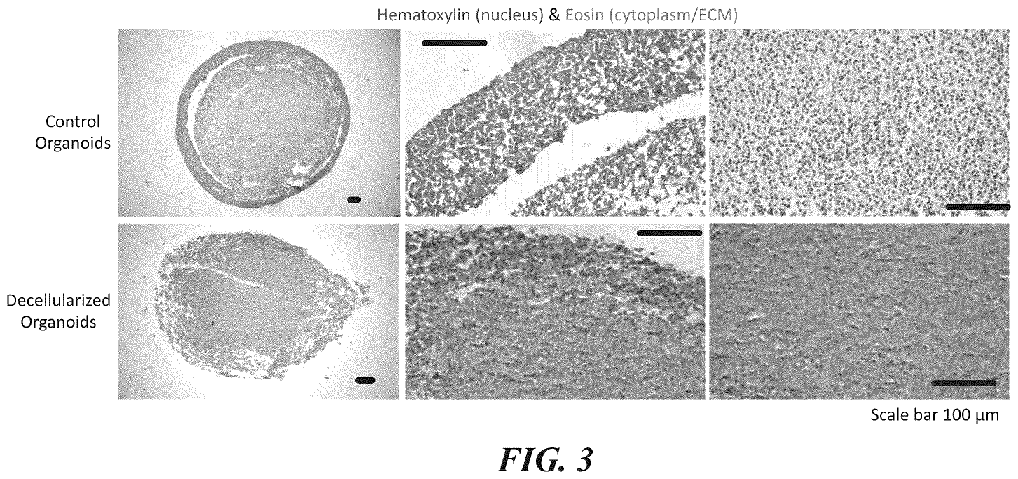

In other implementations, a method of synthesizing an extracellular matrix is disclosed. The method comprises culturing a plurality of organoids then decellularizing the plurality of organoids to obtain the extracellular matrix. FIG. 3 shows the resulting ECM after decellularization.

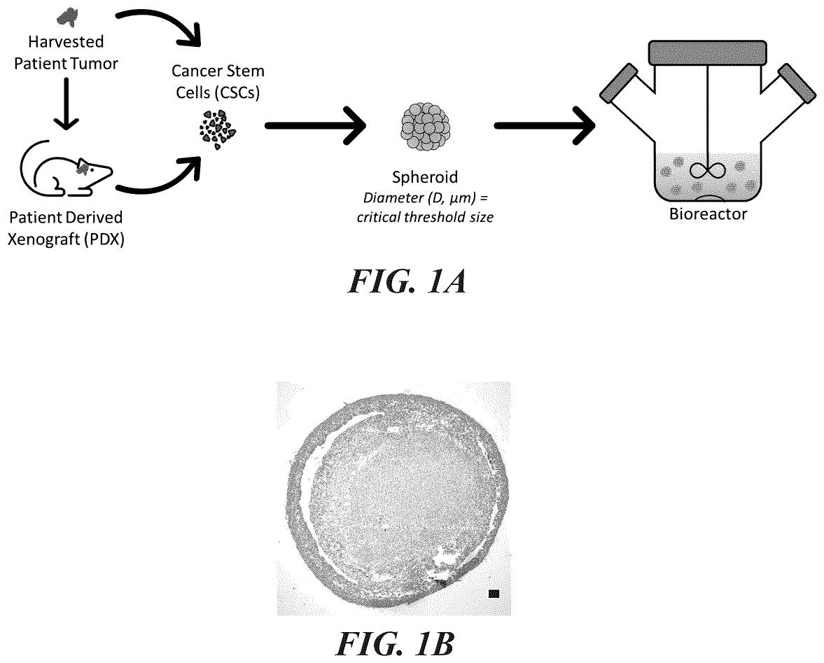

The organoids may be cultured according to the method shown in FIG. 1A, wherein precursor cells are harvested from a source, such as cancer stem cells or patient derived Xenograft (PDX). A spheroid of the precursor cells is induced to grow until a critical threshold size. The spheroid is formed without the use of a scaffolding matrix, such as commonly used Matrigel, or induced differentiation. The critical threshold size is determined when the spheroid responds to its physical environment and exhibits self-differentiation.

The critical threshold size may be from about 100 μm to about 600 μm. In particular, the critical threshold size may be about 150 μm, about 200 μm, about 250 μm, about 300 μm, about 350 μm, about 400 μm, about 450 μm, about 500 μm, or about 550 μm.

Once the spheroids reach the critical threshold size, a plurality of spheroids are inoculated in a bioreactor and exposed to continuous mechanical stress. In one aspect, a plurality of spheroids may be an optimal number of spheroids for the bioreactor vessel size. In some aspects, the method may be applied to batch processing of spheroids to obtain reproducible, spherical organoids, as shown in FIG. 1B.

The mechanical stress may be shear stress and/or agitation. In one aspect, the mechanical stress is shear stress and agitation. In some implementations, the mechanical stress may be achieved by use of magnetic stirring and/or stirring with a baffle having a single blade or multiple blades. In one implementation, the mechanical stress is achieved by use of magnetic stirring and a single blade baffle.

In some aspects, the precursor cells are exposed to shear stress from about 0.1 Pa to about 0.7 Pa. In particular, the precursor cells are exposed to shear stress of about 0.2 Pa, about 0.3 Pa, about 0.4 Pa, about 0.5 Pa, or about 0.6 Pa.

In some aspects, the precursor cells are exposed to agitation having a rate of about 60 rpm to about 120 rpm. In particular, the precursor cells are exposed to agitation having a rate of about 70 rpm, about 80 rpm, about 90 rpm, about 100 rpm, or about 110 rpm.

In some implementations, the bioreactor has a Reynolds number of about 600 to about 2200. In particular, the bioreactor has a Reynolds number of about 800, about 1000, about 1200, about 1400, about 1600, about 1800, or about 2000.

The organoids are allowed to grow in the bioreactor until the organoids reach a diameter of at least 1 mm. The mechanical stress is continuously applied in the bioreactor until the desired organoid diameter is obtained. The organoids may be transferred to a new bioreactor following clean-in-place protocols in order to prevent fouling. During organoid growth in the bioreactor, care is taken not to overcrowd the bioreactor.

In some aspects, the produced organoids may have a diameter of about 400 μm to about 5 mm. In particular, the produced organoids have a diameter of about 600 μm, about 800 μm, about 1 mm, about 1.5 mm, about 2 mm, about 2.5 mm, about 3 mm, about 3.5 mm, about 4 mm, or about 4.5 mm.

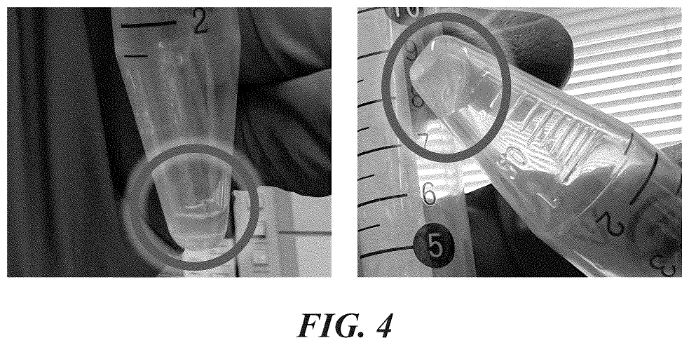

In yet other implementations, a method of forming an extracellular matrix-based hydrogel is disclosed. The method may include culturing an organoid, decellularizing the organoid to obtain an ECM (per FIG. 3), then digesting and neutralizing the ECM to form the hydrogel (per FIG. 4).

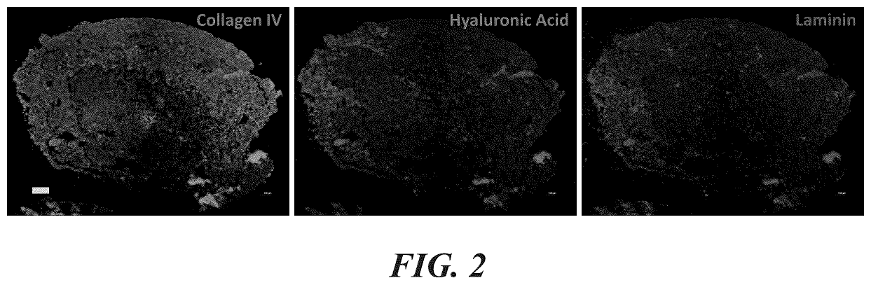

In some aspects, the organoids are human-derived organoids, in particular, the organoids may comprise glioblastoma organoids. The organoids may be chosen to produce the desired ECM, such as a collagen IV, which is present in brain stem cells, hyaluronic acid, or laminin, as shown in FIG. 2.

In some implementations, the method to form the ECM or hydrogel may further comprise post-decellularization processing of the extracellular matrix. Post-decellularization processes may be modifications to the physical form, crosslinking, and/or sterilization of the ECM.

### Example ECM Quantification and Immunohistochemical Visualization

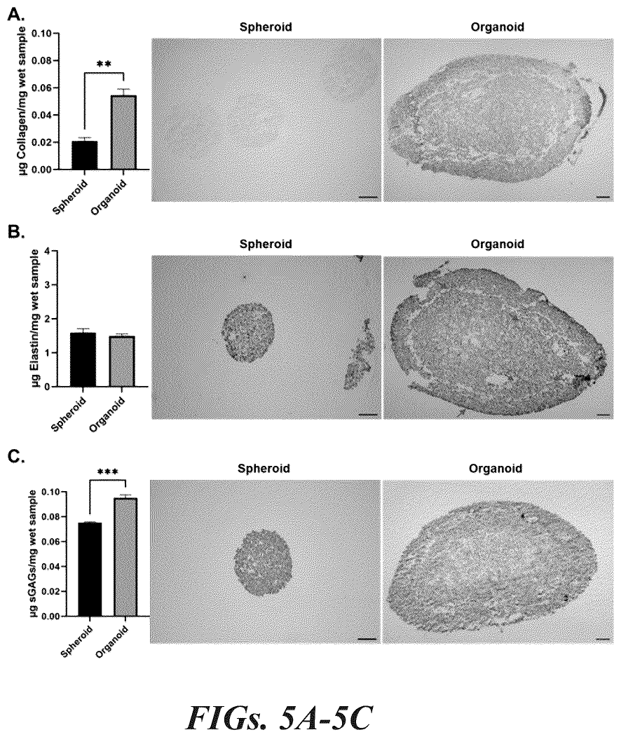

Extracellular matrix (ECM) quantification and immunohistochemical visualization of glioblastoma multiform spheroids and approximately 2.0 mm organoids from the patient derived xenograft cell line, JX6, for collagen (FIG. 5A), elastin (FIG. 5B), and glycosaminoglycans (GAGs) (FIG. 5C) are presented. Collagen quantification was performed using a hydroxyproline-based quantification kit and stained with picrosirius red such that red indicates total collagen, yellow indicates collagen type I, and green indicates collagen type III (FIG. 5A). Elastin was quantified using a 5,10,15,20-tetraphenyl-21H,23H-porphine tetrasulfonate (TPPS)-based kit and stained with Verhoff's Van Gieson such that blue-black to black indicates elastin fibers, blue to black indicates nuclei, red indicates collagen, and yellow indicates other tissue elements. The arrows are pointed towards more highly expressed elastin within the organoid (FIG. 5B). Sulfated GAGs (sGAGs) were quantified using a dimethylmethylene blue (DMMB)-based quantification kit and stained with alcian blue 8GX counterstained with Nuclear Fast Red. Alcian blue and Nuclear Fast red together create a purple color to indicate GAGs, red indicates cell nuclei, and pale pink indicates cell cytoplasm (FIG. 5C). ECM quantification was normalized to wet sample mass.

Total collagen was quantified utilizing a hydroxyproline-based Sensitive Tissue Collagen Assay kit (QZBTISCOL 1, QuickZyme) and visualized using a Picrosirius Red Stain Kit (24901-250, Polysciences) without nuclear staining according to manufacturer protocols. The red staining indicates general collagen; yellow indicates collagen type I; and green indicates collagen type III. Organoids with a diameter of approximately 2.0 mm have significantly more collagen present than spheroids. The collagen is more universally expressed than spatially expressed in the organoids, suggesting that collagen production is size dependent (FIG. 5A).

Elastin was quantified utilizing 5,10,15,20-tetraphenyl-21H,23H-porphine tetrasulfonate (TPPS)-based Fastin™ Elastin Assay kit (F2000, Biocolor) and visualized through the Verhoffs Van Geison staining (26374-Series, Electron Microscopy Science) according to manufacturer protocols. The elastin fibers were stained blue-black to black; nuclei were stained blue to black; collagens were stained red; and other tissue elements were stained yellow. Both the spheroids and organoids indicated collagen expression closer to the perimeter. Elastin was universally expressed in the spheroids but expressed more in some regions as indicated about the arrows in FIG. 5B, suggesting elastin production may be dependent more on spatial cellular composition within organoids.

Sulfated glycosaminoglycans (sGAGs) were quantified utilizing a dimethylmethylene blue (DMMB)-based Blyscan™ Glycosaminoglycan Assay kit (B1000, Biocolor) and visualized through alcian blue 8GX in 3% acetic acid, pH 2.5 (AAJ6012214, Fisher Scientific) with a Nuclear Fast Red (AAJ61010AP, Fisher Scientific) nuclear counterstain according to manufacturer protocols. Alcian blue stains GAGs and hyaluronic acid blue to purple while Nuclear Fast red stains cell nuclei red and cytoplasm pale pink. Organoids had significantly more sGAGs present than spheroids. It was difficult to identify any GAG expression in the spheroids. However, organoids appeared to express GAGs more towards the center (FIG. 5C). This suggests that GAG production may be dependent on spatial cellular composition and size. An approximate 26% increase of ECM in the organoids compared to the spheroids was observed from the total amount of ECM (collagen, elastin, and sGAGs) determined from the quantification for the spheroid and organoids.

In summary, there's a baseline expression of relative amounts of ECM in spheroids. The relative amount of ECM either remained the same and was more spatially expressed, and/or increased with size when comparing spheroids and organoids. The overall increased amount and heterogeneity of ECM in the organoids compared to the spheroids further supports why organoid-derived hydrogels are a perfect candidate for human-derived hydrogel production.

What is claimed is:

1. A hydrogel comprising an extracellular matrix;
wherein the extracellular matrix comprises decellularized organoids, wherein the organoids are derived from human stem cells.

2. The hydrogel of claim 1, wherein the human stem cells comprise human brain stem cells.

3. The hydrogel of claim 1, wherein the human stem cells comprise glioblastoma stem cells.

4. The hydrogel of claim 1, wherein the extracellular matrix if further digested and neutralized to form the hydrogel.

5. The hydrogel of claim 4, wherein the extracellular matrix retains a high degree of heterogeneity.

6. The hydrogel of claim 1, wherein the organoids comprise glioblastoma organoids.

7. The hydrogel of claim 1, wherein the extracellular matrix is additionally modified (e.g physical form, crosslinking, sterilization).

8. A method of synthesizing an extracellular matrix comprising:
a) culturing a plurality of organoids; and
b) decellularizing the plurality of organoids to obtain the extracellular matrix.

9. The method of claim 8, wherein the organoids comprise human organoids.

10. The method of claim 8, wherein the organoids comprise glioblastoma organoids.

11. The method of claim 8, further comprising post-decellularization processing of the extracellular matrix.

12. The method of claim 11, wherein post-decellularization processing comprises modifications to a physical form of the extracellular matrix.

13. The method of claim 11, wherein post-decellularization processing comprises additional crosslinking of the extracellular matrix.

14. The method of claim 11, wherein post-decellularization processing comprises sterilization of the extracellular matrix.

15. The method of claim 8, further comprising polymerizing the extracellular matrix to form a hydrogel.

16. A method for regenerating tissue, the method comprising administering comprising an extracellular matrix, wherein the extracellular matrix comprises decellularized organoids, wherein the organoids are derived from human stem cells.

17. The method of claim 16, wherein the tissue is regenerated in vivo.

18. The method of claim 16, wherein the tissue is regenerated in vitro.
